(19) United States
(12) Patent Application Publication
(10) Pub. No.: US 2010/0221200 A1
(43) Pub. Date: Sep. 2, 2010

(54) COMPOSITIONS AND METHODS FOR SKIN CARE

(75) Inventor: Matthew J. During, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 12/669,246

(22) PCT Filed: Jul. 17, 2008
(86) PCT No.: PCT/US08/70313

§ 371 Date: May 11, 2010

Related U.S. Application Data
(60) Provisional application No. 60/950,224, filed on Jul. 17, 2007.

(51) Int. Cl. A61K 8/64; A61Q 17/04; A61Q 19/00

(52) U.S. Cl. 424/59; 514/16

(57) ABSTRACT

The present disclosure contemplates compositions and methods for skin care. In some embodiments, the compositions may comprise delta opioid receptor agonists. The skin care composition may comprise peptide delta opioid receptor agonists, non-peptide delta opioid receptor agonists, or combinations thereof. In further embodiments, the peptide delta opioid receptor agonists may be chosen from enkephalins and/or deltorphins. In yet other embodiments, the delta opioid receptor agonists may be conjugated to another molecule. For example, the delta opioid receptor agonist may be conjugated to a lipid in order to facilitate entry across the stratum corneum. Further disclosed herein is the cosmetic use of at least one combination as described above, in a composition suitable for topical application to the skin, as an agent for smoothing out wrinkles and fine lines, such as expression wrinkles.

### CROSS REFERENCE TO RELATED APPLICATIONS

This application claims priority to and any other benefit of U.S. Provisional Application Ser. No. 60/950,224, filed on Jul. 17, 2007, the entire content of which is hereby incorporated by reference herein.

### BACKGROUND

Changes in the structure and function of the skin, particularly marked in exposed skin, include changes in the thickness of specific skin layers, the loss of elastin, altered collagen, and reduced skin epidermal cell turnover. All of these changes result in the cosmetic signs of aging (including wrinkles, loss of elasticity, stretch marks, uneven pigment, loss of hydration). As advances in medicine succeed in reducing the burden of fatal disease, particularly cardiovascular diseases and cancer, and with the mean life expectancy ever increasing, there is a much greater focus now on esthetic or appearance medicine.

Even people in their late middle age (and what years constitute middle age is continuously revised upwards) are fit and healthy, have great vitality, yet their physical appearance and particularly the inevitable photodamage on the face, neck, hands and other exposed skin remains an indicator as to their true chronological age. Such individuals, particularly women, but increasingly men also, attempt to use many products to reverse these sun and aging associated changes.

As a result, the skin care market is large and growing, but it is full of products of dubious merit but creative marketing. The “active” components of these products included antioxidant vitamins (C and E), other antioxidants typically of natural origin, as well as extracts or compounds isolated from seeds, flowers, or other components of plants, derived from marine life, phytoestrogens (genistein, diadzein), copper peptides, elastin and collagen fragments, and various growth factors. Amongst those products that really do seem to improve the appearance of the skin, and have multiple controlled clinical trials to prove it, are retinoic acid (Retin-A), and chemical peels (glycolic acid). Beyond these products are thousands of claims but little science. Both retinoic acid and chemical peels have considerable limitations and adverse events associated with their use, so there is a need to come up with much safer alternatives, including those that may have superior or complementary activity.

### SUMMARY

Provided herein are compositions and methods for skin care. In some embodiments, the compositions may comprise delta opioid receptor agonists. In other embodiments, the compositions may comprise peptide delta opioid receptor agonists, non-peptide delta opioid receptor agonists, or combinations thereof. The compositions may be pharmaceutical or cosmetic compositions. In some embodiments, the compositions may be applied in any suitable manner. In other embodiments, the compositions may be applied topically.

The compositions may comprise at least one compound that increases the amount of delta opioid receptor a cell produces. These compositions may be pharmaceutical or cosmetic compositions. In some embodiments, these compositions may be applied in any suitable manner. In other embodiments, these compositions may be applied topically.

Also provided herein are methods for skin care. The method may comprise delivering a cosmetically effective amount of at least one delta opioid receptor agonist. In some embodiments, the method may comprise delivering a cosmetically effective amount of at least one peptide delta opioid receptor agonist, non-peptide delta opioid receptor agonist, or combinations thereof. In other embodiments, the method may comprise delivering a therapeutically effective amount of at least one delta opioid receptor agonist. In yet other embodiments, the method may comprise delivering a cosmetically effective amount of at least one substance that increases the amount of delta opioid receptor a cell produces.

A method of identifying delta opioid receptor agonists suitable for skin care is also contemplated herein. In some embodiments, the method may comprise a step of using a delta opioid receptor, a truncated delta opioid receptor, a molecule sufficiently similar to a delta opioid receptor, or any combination thereof to identify agonist molecules suitable for skin care.

Additional features and advantages will be set forth in part in the description that follows, and in part will be obvious from the description, or may be learned by practice of the embodiments disclosed. The objects and advantages of the embodiments will be realized and attained by means of the elements and combinations particularly pointed out in the appended claims.

It is to be understood that both the foregoing general description and the following detailed description are exemplary and explanatory only and are not restrictive of the embodiments, as claimed.

### DETAILED DESCRIPTION

The compositions and methods for skin care will now be described by reference to some more detailed embodiments. The compositions and methods may, however, be embodied in different forms and should not be construed as limited to the embodiments set forth herein. Rather, these embodiments are provided so that this disclosure will be thorough and complete, and will fully convey the scope of the compositions and methods to those skilled in the art.

Unless otherwise defined, all technical and scientific terms used herein have the same meaning as commonly understood by one of ordinary skill in the art. The terminology used in the description herein is for describing particular embodiments only and is not intended to be limiting of the embodiments. As used in the description and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the embodiments are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The present disclosure contemplates compositions and methods for skin care. In some embodiments, the compositions may comprise delta opioid receptor agonists. There are three classes of opioid receptors: mu (μ); kappa (κ); and delta (δ). The mu receptor mediates the action of the potent analgesics and drugs of abuse, including heroin and morphine. Kappa receptors are widely distributed in the brain, spinal cord, and in pain neurons. Dynorphin is the major endogenous agonist of the kappa receptor, and there are no highly selective kappa agonists used clinically. Similarly, there are no delta agonists currently in common clinical use, although many of the mu agonists have partial activity on the other receptor classes. However, the mu receptor agonists come with severe side effects, as would be expected from potent analgesics and drugs of abuse, and therefore would not be suitable for skin care purposes.

The delta opioid receptor is of particular interest as the phenomenon of hibernation is in believed to be delta receptor mediated. If the delta opioid receptor agonist, DADLE, is administered to summer-active ground squirrels it induces hibernation. Hibernation is of major interest in the study of aging, and age-related changes in the structure and function of tissues including the skin, because studies have shown that longevity in hibernating mammals like hamsters is directly proportional to the amount of time spent in hibernation. In general, hibernation, via the energy consumption theory of aging, is believed to improve the signs of aging akin to how keeping a car in the garage will reduce signs of the car's aging as opposed to a car with high miles that would show the wear and tear that comes with age. Moreover, the delta opioid receptor agonist DADLE also improves the viability and preservation of isolated or transplanted organs including heart, lung and kidneys. While not wishing to be bound by any particular theory, it is believed that delta opioid receptor agonists may improve the texture and/or appearance of the surface to which it is applied, for example the skin, without necessarily rendering a benefit or an effect of treating or preventing an abnormal biological condition or a disease, or may improve the texture and/or appearance of the surface to which it is applied, for example the skin, by rendering a benefit or an effect of treating or preventing an abnormal biological condition or a disease.

Delta opioid receptors from multiple species are known and could be used to study the effects of agonists, including for example human (Genbank accession no.: NM-000911), rat (Genbank accession no.: U00475), mouse (Genbank accession no.: L11064), zebrafish (Genbank accession no.: NM-131258), cow, bass, shark, and frog. Furthermore, partial sequences of the delta opioid receptor from monkey (Genbank accession no.: PC2218) and pig (Genbank accession no.: U71149) are also known. In some embodiments, the delta opioid receptor may be glycosylated and/or amidated and/or lipidated. In some embodiments, the delta opioid receptor may be truncated, for example, amino acids from the N-terminus, C-terminus, or both may be removed.

It is noted that mutations or modifications may be made to the delta opioid receptor described herein without any effect on the disclosed methods or compositions for skin care. In some embodiments, the delta opioid receptor may comprise additions, deletions, insertions, mutations, or combinations thereof The types of mutations that may be made are of various types. Deletion mutations, in which certain nucleotide bases are deleted, form a polypeptide sequence resulting in deletions or changes in amino acids in the translated polypeptide. Insertion mutations occur via the addition of a nucleotide base within a given coding sequence resulting in frame shift of the polynucleotide sequence. And mutations that result in substitutions of one amino acid for another can also be made.

With regard to amino acid substitutions, a variety of amino acid substitutions can be made. Amino acids can generally be grouped as follows: (1) amino acids with non-polar or hydrophobic side groups (A, V, L, I, P, F, W, and M); (2) amino acids with uncharged polar side groups (G, S, T, C, Y, N, and Q); (3) polar acidic amino acids, negatively charged at pH 6.0-7.0 (D and E); and (4) polar basic amino acids, positively charged at pH 6.0-7.0 (K, R, and H). Generally, “conservative” substitutions, i.e., those in which an amino acid from one group is replaced with an amino acid from the same group, can be made without an expectation of impact on activity. Further, some non-conservative substitutions may also be made without affecting activity. Those of ordinary skill in the art will understand what substitutions can be made without impacting activity.

While the naturally occurring amino acids are discussed above, non-naturally occurring amino acids, or modified amino acids, are also contemplated and may be used as substitutions in the recited delta opioid receptors. Thus, as used herein, “amino acid” refers to natural amino acids, non-naturally occurring amino acids, and amino acid analogs, all in their D and L stereoisomers. Natural amino acids include alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), serine (S), threonine (T), tryptophan (W), tyrosine (Y) and valine (V). Non-naturally occurring amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4 diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine, and pipecolic acid.

In some embodiments, the delta opioid receptor may be a fusion protein, for example, an amino acid sequence may be in communication with the N-terminus, C-terminus, interior portion of the receptor, or any combinations thereof. In some embodiments, agonists of any or all of the above and below described delta opioid receptors may be used for delivery to a subject in need of such delivery.

Agonists are molecules that interact with receptor molecules. Some agonists are capable of binding to a receptor to trigger a response. Various types of responses can be triggered by agonist binding, including but not limited to cellular responses. Agonists may also mimic the action of an endogenous ligand (such as hormone or neurotransmitter, for example) that binds to the same receptor. In some embodiments, the composition may comprise peptide delta opioid receptor agonists, non-peptide delta opioid receptor agonists, or combinations thereof. In further embodiments, the peptide delta opioid receptor agonists may be chosen from enkephalins and/or deltorphins In still further embodiments, the peptide delta opioid receptor agonists may be chosen from at least one of Deltorphin (Tyr-D-Met-Phe-His-Leu-Met-Asp-NH₂), D-Ala²-Deltorphin I (Tyr-D-Ala-Phe-Asp-Val-Val-Gly-NH₂), D-Ala²-Deltorphin II (Tyr-D-Ala-Phe-Glu-Val-Val-Gly-NH₂), D-Leu²-Deltorphin (Tyr-D-Leu-Phe-Ala-Asp-Val-Ala-Ser-Thr-Ile-Gly-Asp-Phe-Phe-His-Ser-Ile-NH₂), DADLE (Tyr-D-Ala-Gly-Phe-D-Leu), DPDPE (Tyr-D-Pen-Gly-Phe-D-Pen), pCl-Phe⁴-DPDPE (Tyr-D-Pen-Gly-p-chloro-Phe-D-Pen), DSLET (Tyr-D-Ser-Gly-Phe-Leu-Thr), AIB, 2-aminotetralin-2-carboxylic acid (Atc), 6-hydroxy-2-aminotetralin-2-carboxylic acid (Hat), biphalin (Tyr-D-Ala-Gly-Phe-NH-NH-Phe-Gly-D-Ala-Tyr), Leu-Enkephalin (H-Tyr-Gly-Gly-Phe-Leu-OH), Met-Enkephalin (H-Tyr-Gly-Gly-Phe-Met-OH), JOM-13 (H-Tyr-c[D-Cys-Phe-D-Pen]-OH), DTLET (Tyr-D-Thr-Gly-Phe-Leu-Thr), DSTBULET (Tyr-D-Ser(OtBu)-Gly-Phe-Leu-Thr), BUBU (Tyr-D-Ser(OtBu)-Gly-Phe-Leu-Thr(OtBu)), BUBUC (Tyr-D-Cys(StBu)-Gly-Phe-Leu-Thr(OtBu)), and salmon-calcitonin. In some embodiments, analogues of the peptide delta opioid receptor agonists may be used, for example, peptide agonists having substitutions, enzymatic, and/or chemical modifications. In some embodiments, the enzymatic modification may result in conversion of L-amino acids to D-amino acids. In some embodiments, the peptide agonists may comprise L or D isomers of any or all amino acids. In some embodiments, the peptide agonist may comprise all D-amino acids, which may result in resistance to proteases. In some embodiments, the peptide agonists may be glycosylated, amidated, lipidated, and/or cyclized. In some embodiments, the peptide agonists may be isolated from a natural source or made synthetically.

In yet other embodiments, the delta opioid receptor agonists may be conjugated to another molecule. For example, the delta opioid receptor agonist may be conjugated to a lipid in order to facilitate entry across the stratum corneum. Various lipids that can be conjugated to delta opioid receptor agonists are known, including but not limited to, saturated and unsaturated lipids. Examples of saturated lipids that may be conjugated to a delta opioid receptor agonist include, but are not limited to acetic, butyric, capronic, caprylic, caprynic, lauric, myristic, palmitic, stearic, arachidic, behenic, and lignoceratic acids. Examples of unsaturated lipids include, but are not limited to palmitolic, oleic, linoleic, γ-linoleic, α-linoleic, eicosadinoic, eicosatrinoic, arachidonic, eicosapentaenoic, docosapentaeoic, and docosahexaenoic acids. In some embodiments, the delta opioid receptor agonist deltorphin II may be conjugated to a saturated or unsaturated lipid. Deltorphin II may be conjugated as follows:

Deltorphin II conjugated to saturated lipids:

CH3CO-Y-D-AFEVVG-NH₂ Acetic

CH3(CH₂)₂CO-Y-D-AFEVVG-NH₂ Butyric

CH3(CH₂)₄CO-Y-D-AFEVVG-NH₂ Capronic

CH3(CH₂)₆CO-Y-DD-AFEVVG-NH₂ Caprylic

CH3(CH₂)₈CO-Y-D-AFEVVG-NH₂ Caprynic

CH3(CH₂)₁₀CO-Y-D-AFEVVG-NH₂ Lauric

CH3(CH₂)₁₂CO-Y-D-AFEVVG-NH₂ Myristic

CH3(CH₂)₁₄CO-Y-D-AFEVVG-NH₂ Palmitic

CH3(CH₂)₁₆CO-Y-D-AFEVVG-NH₂ Stearic

CH3(CH₂)₁₈CO-Y-D-AFEVVG-NH₂ Arachidic

CH3(CH₂)₂₀CO-Y-D-AFEVVG-NH₂ Behenic

CH3(CH₂)₂₂CO-Y-D-AFEVVG-NH₂ Lignoceratic

Deltorphin II conjugated to unsaturated lipids:

CH3(CH₂)₅CH═CH(CH₂)₇COO-Y-D-AFEVVG-NH₂ Palmitoleic

CH3(CH₂)₇CH═CH(CH₂)₇COO-Y-D-AFEVVG-NH₂ Oleic

CH3(CH₂)₄(CH═CHCH₂)₂(CH₂)₆COO-Y-D-AFEVVG-NH₂ Linoleic

CH3(CH₂)₄(CH═CHCH₂)₃(CH₂)₃COO-Y-D-AFEVVG-NH₂ γ-Linoleic

CH3(CH₂)(CH═CHCH₂)₃(CH₂)₆COO-Y-D-AFEVVG-NH₂ α-Linoleic

CH3(CH₂)₄(CH═CHCH₂)₂(CH₂)₈COO-Y-D-AFEVVG-NH₂ Eicosadinoic

CH3(CH₂)₄(CH═CHCH₂)₃(CH₂)₅COO-Y-D-AFEVVG-NH₂ Eicosatrinoic

CH3(CH₂)₄(CH═CHCH₂)₄(CH₂)₂COO-Y-D-AFEVVG-NH₂ Arachidonic

CH3(CH₂)(CH═CHCH₂)₅(CH₂)₂COO-Y-D-AFEVVG-NH₂ Eicosapentaenoic

CH3(CH₂)(CH═CHCH₂)₅(CH₂)₄COO-Y-D-AFEVVG-NH₂ Docosapentaeoic

CH3(CH₂)(CH═CHCH₂)₆(CH₂)COO-Y-D-AFEVVG-NH₂ Docosahexaenoic

In some embodiments, the non-peptide delta opioid receptor agonists may be chosen from at least one of SNC80, SNC86, SNC162, TAN-67, opioid receptor specific diarylmethylpiperazine compounds, opioid receptor specific diarylmethylpiperidine compounds, and BW373U86. In further embodiments, the non-peptide agonists may be modified, for example, chemically or enzymatically modified.

In some embodiments, the compositions may be pharmaceutical and/or cosmetic compositions. A cosmetic composition may improve the texture and/or appearance of the surface to which it is applied, for example the skin, without necessarily rendering a benefit or an effect of treating or preventing an abnormal biological condition or a disease. A pharmaceutical composition may improve the texture and/or appearance of the surface to which it is applied, for example the skin, by rendering a benefit or an effect of treating or preventing an abnormal biological condition or a disease. In some embodiments, the compositions for skin care may be used to prevent, retard, arrest, treat, and/or reverse the process of skin atrophy in mammalian skin. Skin atrophy may be the thinning and/or general degradation of the dermis sometimes characterized by a decrease in collagen and/or elastin as well as the decreased number, size, and/or doubling potential of fibroblast cells Skin atrophy is a natural result of aging, but may also be caused by intrinsic and/or extrinsic factors such as natural chronoaging, photodamage, burns, or chemical damage. In some embodiments, the compositions for skin care may be used to treat dry skin, dandruff, acne, keratoses, psoriasis, eczema, pruritis, age spots, lentigines, melasmas, wrinkles, warts, blemished skin, hyperpigmented skin, hyperkeratotic skin, inflammatory dermatoses, age-related skin changes, and/or skin in need of cleansers.

In some embodiments, the compositions for skin care may be used in a safe and effective amount. A safe and effective amount may be an amount of compound or composition sufficient to induce a positive modification in the condition to be treated, but low enough to avoid serious side effects (at a reasonable benefit/risk ratio), within the scope of sound medical judgment. The safe and effective amount of the compound or composition will vary with the particular condition being treated, the age and physical condition of the patient being treated, the severity of the condition, the duration of the treatment, the nature of concurrent therapy, the specific compound, compounds or composition employed, the particular pharmaceutically-acceptable carrier utilized, and like factors within the knowledge and expertise of the attending physician or health care provider.

The compositions for skin care disclosed herein may comprise about 0.00001% to about 50% of delta opioid receptor agonist. In some embodiments, the compositions may comprise about 0.0001% to 50%, 0.001% to 50%, 0.01% to 50%, 0.1% to 50% , 0.2% to 50%, 0.3% to 50%, 0.4% to 50%, 0.5% to 50%, 0.6% to 50%, 0.7% to 50%, 0.8% to 50%, 0.9% to 50%, 1% to 50%, 2% to 50%, 3% to 50%, 4% to 50%, 5% to 50%, 6% to 50%, 7% to 50%, 8% to 50%, 9% to 50%, 10% to 50%, 20% to 50%, 30% to 50%, 0.01% to 40%, 0.01% to 30%, 0.01% to 20%, 0.01% to 10%, 0.01% to 9%, 0.01% to 8%, 0.01% to 7%, 0.01% to 6%, 0.01% to 5%, 0.01% to 4%, 0.01% to 3%, 0.01% to 2%, 0.01% to 1%, or 0.01% to 0.1% of delta opioid receptor agonist. It should be understood that two or more delta opioid receptor agonists can be used in combination such that the combined % of those agonists used in the above-mentioned compositions is within those ranges mentioned above. Such compositions may be applied topically, so as to minimize systemic effects or undesirable side effects. The compositions may also be employed in pharmaceutical compositions suitable for parenteral (including subcutaneous, transdermal, intramuscular and intravenous) administration, although the most suitable route in any case will depend on the nature and severity of the condition being treated. The mode of administration for treating skin disorders, in particular skin atrophy or the skin disorders described above, is topical. In addition, the compositions may be further employed in cosmetic compositions. In such an instance, the mode of administration for treating skin disorders is topical. In some embodiments, the compositions may be compatible with the skin and its integuments, for example eyelashes, nails, and hair, and/or mucous membranes.

The delta opioid receptor agonist compositions may comprise the below cosmetic additives. Cosmetic additives may be added to achieve a desired cosmetic result. Desired cosmetic results may be determined by one of ordinary skill in the art or the user of the disclosed compositions. Cosmetic additives may include, but are not limited to, carriers, excipients, vehicle ingredients, moisturizers, humectants, pharmaceutical compositions, cosmetic compositions, pharmaceutical salts, cosmetic salts, adjuvants, oils, emulsifiers, co-emulsifiers, gelling agents, absorbers, solvents, photoprotective agents, and inert bases.

The compositions useful for topical application may contain additional ingredients such as carrier, excipient, or vehicle ingredients such as, for example, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments which are non-toxic and pharmaceutically or dermatologically acceptable. Additionally, moisturizers or humectants can be added to the present compositions if desired. Examples of such additional ingredients can be found in Remington's Pharmaceutical Sciences, Eighteenth Edition, A. R. Gennaro, Ed., Mack Publishing Co., Easton, Pa., 1990.

In addition to these and other vehicles which will be obvious to those of ordinary skill in the art, it will be understood that the pharmaceutical or cosmetic compositions may include other ingredients such as those that improve or eradicate age spots, keratoses and wrinkles; analgesics; anesthetics; antiacne agents; antibacterials; antiyeast agents; antifungal agents; antiviral agents; antidandruff agents; antidermatitis agents; antipruritic agents; antiemetics; antimotion sickness agents; antiinflammatory agents; antihyperkeratolytic agents; antidryskin agents; antiperspirants; antipsoriatic agents; antieborrheic agents; hair conditioners and hair treatment agents; antiaging antiwrinkle agents; antiasthmatic agents and bronchodilators; sunscreen agents; antihistamine agents; skin lightening agents; depigmenting agents; vitamins; corticosteroids; tanning agents; hormones; retinoids; topical cardiovascular agents; clotrimazole; ketoconazole; miconazole; griseofulvin; hydroxyzine; diphenhydramine; pramoxine; lidocaine; procaine; mepivacaine; monobenzone; erythidocaine; procaine; mepivacaine; monobenzone; erythromycin; tetracycline; clindamycin; meclocyline; hydroquinone; minocycline; naproxen; ibuprofen; theophylline; cromolyn; albuterol; retinoic acid; 13-cis retinoic acid; hydrocortisone; hydrocortisone 21-acetate; hydro-cortisone 17-valerate; hydrocortisone 17-butyrate; betamethasone valerate; betamethasone dipropionate; triamcinolone acetonide; fluocinonide; clobetasol propionate; benzoyl peroxide; crotamiton; propranolol; promethazine; vitamin A palmitate; vitamin E acetate and mixtures thereof.

In some embodiments, the compositions can be used as their pharmaceutically or cosmetically acceptable salts. Such salts include, but are not limited to, sodium, potassium, lithium, calcium, magnesium, iron and zinc salts.

This composition may be in any presentation form normally used in cosmetics, and it may, for example, be in the form of an optionally gelled aqueous solution, a dispersion of the lotion type, optionally a two-phase lotion, an emulsion obtained by dispersing a fatty phase in an aqueous phase (O/W emulsion) or conversely (W/O emulsion), or a triple emulsion (W/O/W or O/W/O emulsion) or a vesicular dispersion of ionic and/or nonionic type. These compositions are prepared according to the usual methods. In one embodiment, a composition in the form of an oil-in-water emulsion is used.

This composition may be more or less fluid and may have the appearance of a white or colored cream, an ointment, a milk, a lotion, a serum, a paste or a mousse. It may optionally be applied in the form of an aerosol. It may also be in solid form, such as in the form of a stick. It may be used as a care product and/or as a makeup product for the skin.

In a known manner, the composition disclosed herein may also comprise at least one adjuvant chosen from adjuvants that are common in cosmetics, such as hydrophilic and lipophilic gelling agents, hydrophilic and lipophilic active agents, preserving agents, antioxidants, solvents, fragrances, fillers, screening agents, pigments, odor absorbers and dyestuffs. The at least one adjuvant is present in an amount ranging, for example, from 0.01% to 20% by weight relative to the total weight of the composition. Depending on its nature, the at least one adjuvant may be introduced into the fatty phase, into the aqueous phase, or into lipid vesicles. In any case, these adjuvants, and also the proportions thereof, will be chosen so as not to harm the desired properties of the combination of anti-wrinkle active agents disclosed herein.

When the composition disclosed herein is an emulsion, the proportion of the fatty phase may range from 5% to 80% by weight such as from 5% to 50% by weight relative to the total weight of the composition. The oils, emulsifiers and co-emulsifiers used in the composition in emulsion form are chosen from those conventionally used in the field under consideration. The emulsifier and co-emulsifier are present in the composition in an amount ranging from 0.3% to 30% by weight such as from 0.5% to 20% by weight relative to the total weight of the composition.

As oils which may be used in this disclosure, mention may be made, for example, of mineral oils (liquid petroleum jelly or hydrogenated polyisobutene), oils of plant origin (avocado oil or soybean oil), oils of animal origin (lanolin), silicone oils (cyclomethicone or dimethicone) and fluoro oils (perfluoropolyethers). Fatty alcohols (cetyl alcohol), fatty acids and waxes (carnauba wax or ozokerite) may also be used as fatty substances.

As examples of emulsifiers and co-emulsifiers that may be used herein, mention may be made, for example, of fatty acid esters of polyethylene glycol such as PEG-100 stearate, and fatty acid esters of glycerol such as glyceryl stearate.

Hydrophilic gelling agents that may be included are, for example, carboxyvinyl polymers (carbomer), acrylic copolymers such as acrylate/alkylacrylate copolymers, polyacrylamides, such as crosslinked polyacrylamido-methylpropane-sulphonic acid, polysaccharides, natural gums and clays, and lipophilic gelling agents that may be mentioned include, for example, modified clays, such as bentones, metal salts of fatty acids, hydrophobic silica and polyethylenes.

In one embodiment, the hydrophilic gelling agent for the composition disclosed herein is chosen from a crosslinked polyacrylamido-methylpropane-sulphonic acid as described in EP 0850642, WO9800094 or the Hostacerin AMPS commercialized by Clariant.

The composition disclosed herein may advantageously further comprise at least one compound as an active agent chosen from: desquamating agents; moisturizers; depigmenting and propigmenting agents; anti-glycation agents; NO-synthase inhibitors; agents for stimulating the synthesis of dermal or epidermal macromolecules and/or for preventing their degradation such as agents for stimulating the synthesis of epidermal macromolecules, such as an extract of beech buds (for example, the product sold by the company Gattefosse under the trade name Gatuline), agents for stimulating collagen synthesis, such as soybean protein hydrolysates (for example, the product sold by the company Coletica under the trade name Phytokine), agents for stimulating elastin synthesis and/or for inhibiting collagen degradation, such as an extract of the alga Macrocystis pyrifera (for example, the product sold by the company Secma under the trade name Kelpadelie) and agents for stimulating glycosaminoglycan synthesis, such as an extract of Saccharomyces cerevisiae (for example, the product sold by the company Cognis under the trade name Cytovitin); agents for stimulating fibroblast and/or keratinocyte proliferation or for stimulating keratinocyte differentiation, for example, a soybean protein extract such as the product sold by the company Cognis under the trade name Eleseryl; dermo-relaxants such as sapogenins and natural extracts, such as extract of Wild Yam; tightening agents such as polymers comprising a polysiloxane skeleton onto which are grafted mixed polymer units from the poly(meth)acrylic acid type and of the polyalkyl (meth)acrylate type, such as those sold by the company 3M under the trade names LO21 and VS80; antipollution agents and/or free-radical scavengers; agents that act on the capillary circulation; and agents that act on the energy metabolism of cells.

The compositions disclosed herein may also comprise at least one agent chosen from UVA-active and UVB-active organic and mineral photoprotective agents (absorbers), which are water-soluble or liposoluble, or even insoluble in the cosmetic solvents commonly used.

The organic photoprotective agents are chosen, for example, from anthranilates; cinnamic derivatives; dibenzoylmethane derivatives; salicylic derivatives; camphor derivatives; triazine derivatives such as those described in documents U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β,-diphenylacrylate derivatives; benzotriazole derivatives; benzalmalonate derivatives; benzimidazole derivatives; imidazolines; bis-benzazolyl derivatives as described in documents EP 669 323 and U.S. Pat. No. 2,463,264; p-aminobenzoic acid (PABA) derivatives; methylenebis (hydroxyphenylbenzotriazole) derivatives as described in documents U.S. Pat. Nos. 5,237,071, 5,166,355, GB 2 303 549, DE 197 26 184 and EP 893 119; screening polymers and screening silicones such as those described, for example, in document WO 93/04665; dimers derived from x-alkylstyrene such as those described in document DE 198 55 649; 4,4-diarylbutadienes as described in documents EP 0 967 200, DE 197 46 654, DE 197 55 649, EP-A-1 008 586, EP 1 133 980 and EP 133 981, and mixtures thereof.

In one embodiment, the organic photoprotective agents are chosen from the following compounds: ethylhexyl salicylate, ethylhexyl methoxycinnamate, octocrylene, phenylbenzimidazolesulphonic acid, benzophenone-3, benzophenone-4, benzophenone-5,4-methylbenzylidenecamphor, terephthalylidenedicamphorsulphonic acid, disodiumphenyldibenzimidazoletetrasulphonate, 2,4,6-tris(diisobutyl 4′-aminobenzalmalonate)-s-triazine, anisotriazine, ethylhexyltriazone, diethylhexylbutamidotriazone, methylenebis(benzotriazolyl)tetramethylbutylphenol, drometrizole trisiloxane, 1,1-dicarboxy(2,2′-dimethylpropyl)-4,4-diphenylbutadiene, and mixtures thereof.

The mineral photoprotective agents are chosen from pigments and nanopigments (mean size of the primary particles generally ranging from 5 nm to 100 nm such as from 10 nm to 50 nm) of coated and uncoated metal oxides, for example, nanopigments of titanium oxide (amorphous or crystallized in rutile and/or anatase form), of iron oxide, of zinc oxide, of zirconium oxide or of cerium oxide, which are all UV-photoprotective agents that are well known per se. Standard coating agents are, for example, alumina and/or aluminium stearate. Such coated or uncoated metal oxide nanopigments are described, for example, in documents EP 518 772 and EP 518 773.

The photoprotective agents are generally present in the compositions disclosed herein in proportions ranging from 0.1% to 20% by weight relative to the total weight of the composition such as from 0.2% to 15% by weight relative to the total weight of the composition.

Further disclosed herein is the cosmetic use of at least one combination as described above, in a composition suitable for topical application to the skin, as an agent for smoothing out wrinkles and fine lines, such as expression wrinkles.

Even further disclosed herein is a cosmetic process for treating wrinkled skin, comprising topically applying to the skin a composition disclosed herein, for example, to the areas of the face or forehead marked with expression wrinkles and/or to individuals with expression wrinkles.

According to one embodiment, the composition is applied to the wrinkles and fine lines lying radially around the mouth and/or the eyes and/or horizontally on the forehead and/or in the space between the eyebrows.

Also disclosed herein are compositions which may comprise at least one compound that increases the amount of delta opioid receptor a cell produces. The delta opioid receptor gene has a 5′ untranslated region (UTR) which may be targeted by at least one compound to increase the level of transcription of the delta opioid receptor gene, thereby resulting in an increased expression of delta opioid receptors. The 5′ UTR may comprise cis, trans, and/or promoter elements that may be targeted to increase transcription of the delta opioid receptor gene. In some embodiments, mimics of the cis, trans, and/or promoter elements may be employed to increase transcription of the delta opioid receptor gene. These compositions may also comprise at least one delta opioid receptor agonist as described above.

Methods for skin care are also disclosed herein. In some embodiments, the method may comprise delivering a cosmetically effective amount of at least one delta opioid receptor agonist to an area requiring such delivery. Delivery may be achieved in any such manner which is suitable for skin care. For example, the method may comprise a step of topically applying the at least one opioid receptor agonist to the skin. The skin to which the at least one delta opioid agonist may be applied may be to wrinkled skin for example. In another embodiment, the method may comprise delivering at least one skin care composition described in this disclosure. In yet another embodiment, the method may comprise delivering a therapeutically effective amount of at least one delta opioid receptor agonist and/or at least one skin care composition disclosed herein.

Methods for identifying delta opioid receptor agonists suitable for skin care are also contemplated. The method may comprise a step of using a delta opioid receptor, or any analogue of a delta opioid receptor disclosed herein, or any combination thereof to identify agonists suitable for skin care.

### EXAMPLE 1

A test male subject (body weight ˜71 kg) received an application of 10 μg deltorphin, constituted in an inert base, to the malar surface on the left side of the face extending from the periorbital region to the mandible. The inert base (without the deltorphin) was applied to the right side of the face but the subject was blind to which cream contained the deltorphin. The cream was applied twice daily, in the morning and night. After 3 weeks, the subject was observed. There was no sign of any irritation, no redness, heat or swelling. The left side of the face had a noticeable improved appearance to the right with smoother, more even tone, fuller, plumper looking skin, with less blemishes. Both fine and moderate wrinkles appeared diminished.

### EXAMPLE 2

A test female subject (˜55 kg) received an application of 1 μg of deltorphin in an inert base to the malar surface of the right side of the face, with an identical inert cream applied to the left in a blinded fashion (the subject was unaware of which cream contained the active peptide). After three weeks of morning and evening application of the cream (no other topical agents were applied), the subject was scored for appearance. Similar to Example 1, a noticeable difference was apparent, this time the improvement observed on the right (the actively treated side). Again, a smoother more even tone, plumper fuller appearance of the skin with increased reflectance, and a marked reduction in wrinkles particularly in the periorbital region.

### EXAMPLE 3

A test female subject (˜61 kg) received an application of 100 μg of deltorphin in an inert base to the malar surface on the right side, with an equal amount of inert base, but no active peptide, applied blindly to the left side of the face. The two creams were applied twice daily, morning and night, for three weeks prior to being observed. As in Examples 1 and 2, there were no signs of any allergy, hypersensitivity or irritation with no redness, no swelling, no heat or pruritus. This subject had claimed the cream on the right side had inadvertently got into her eye on one occasion without any significant eye irritation or redness. As in Example 2, the right side of the face showed significant and visible improvements compared to the left side. Here, there was improved skin tone and texture, less blemishes, and readily notable improvements and diminishment in wrinkles, both fine and deep.

What is claimed is:

1. A composition comprising at least one delta opioid receptor agonist and at least one cosmetic additive.

2. The composition according to claim 1, wherein the at least one delta opioid receptor agonist is chosen from peptide delta opioid receptor agonists, non-peptide delta opioid receptor agonists, and combinations thereof.

3. The composition according to claim 2, wherein the at least one delta opioid receptor agonist is a peptide delta opioid receptor agonist.

4. The composition according to claim 3, wherein the peptide delta opioid receptor agonist is chosen from enkephalins and deltorphins.

5. The composition according to claim 3, wherein the peptide delta opioid receptor agonist is chosen from deltorphin, D-Ala²-Deltorphin I, D-Ala²-Deltorphin II, D-Leu²-Deltorphin, DADLE, DPDPE, pCl-Phe⁴-DPDPE, DSLET, AIB, 2-aminotetralin-2-carboxylic acid (Atc), 6-hydroxy-2-aminotetralin-2-carboxylic acid (Hat), biphalin, Leu-Enkephalin, Met-Enkephalin, JOM-13, DTLET, DSTBULET, BUBU, BUBUC, salmon-calcitonin, and combinations thereof.

6. The composition according to claim 3, wherein the peptide delta opioid receptor agonist is enzymatically modified.

7. The composition according to claim 6, wherein the enzymatic modification is chosen from glycosylation, amidation, lipidation, cyclization, and combinations thereof.

8. The composition according to claim 3, wherein the peptide delta opioid receptor agonist is chemically modified.

9. The composition according to claim 8, wherein the chemical modification is chosen from glycosylation, amidation, lipidation, cyclization, and combinations thereof.

10. The composition according to claim 9, wherein the chemical modification is lipidation.

11. The composition according to claim 10, wherein the lipidation is chosen from conjugation of saturated and unsaturated lipids.

12. The composition according to claim 11, wherein the saturated lipid is chosen from acetic, butyric, capronic, caprylic, caprynic, lauric, myristic, palmitic, stearic, arachidic, behenic, lignoceratic acid, and combinations thereof.

13. The composition according to claim 11, wherein the unsaturated lipid is chosen from palmitoleic, oleic, linoleic, y-linoleic, a-linoleic, eicosadinoic, eicosatrinoic, arachidonic, eicosapentaenoic, docosapentaeoic, docosahexaenoic acid, and combinations thereof.

14. The composition according to claim 2, wherein the at least one delta opioid receptor agonist is a non-peptide delta opioid receptor agonist.

15. The composition according to claim 14, wherein the non-peptide delta opioid receptor agonist is chosen from SNC80, SNC86, SNC162, TAN-67, opioid receptor specific diarylmethylpiperazine compounds, opioid receptor specific diarylmethylpiperadine compounds, BW373U86, and combinations thereof

16. The composition according to claim 1, wherein the at least one cosmetic additive is chosen from carriers, excipients, vehicle ingredients, moisturizers, humectants, pharmaceutical compositions, cosmetic compositions, pharmaceutical salts, cosmetic salts, adjuvants, oils, emulsifiers, co-emulsifiers, gelling agents, absorbers, solvents, photoprotective agents, inert bases, and mixtures thereof.

17. The composition according to claim 16, wherein the excipient is chosen from water, acetone, ethanol, ethylene glycol, propylene glycol, butane-1,3-diol, isopropyl myristate, isopropyl palmitate, mineral oil, and mixtures thereof.

18. The composition according to claim 17, wherein the pharmaceutical and cosmetic compositions are chosen from ingredients such as those that improve or eradicate age spots, keratoses and wrinkles; analgesics; anesthetics; antiacne agents; antibacterials; antiyeast agents; antifungal agents; antiviral agents; antidandruff agents; antidermatitis agents; antipruritic agents; antiemetics; antimotion sickness agents; antiinflammatory agents; antihyperkeratolytic agents; antidryskin agents; antiperspirants; antipsoriatic agents; antieborrheic agents; hair conditioners and hair treatment agents; antiaging antiwrinkle agents; antiasthmatic agents and bronchodilators; sunscreen agents; antihistamine agents; skin lightening agents; depigmenting agents; vitamins; corticosteroids; tanning agents; hormones; retinoids; topical cardiovascular agents; clotrimazole; ketoconazole; miconazole; griseofulvin; hydroxyzine; diphenhydramine; pramoxine; lidocaine; procaine; mepivacaine; monobenzone; erythidocaine; procaine; mepivacaine; monobenzone; erythromycin; tetracycline; clindamycin; meclocyline; hydroquinone; minocycline; naproxen; ibuprofen; theophylline; cromolyn; albuterol; retinoic acid; 13-cis retinoic acid; hydrocortisone; hydrocortisone 21-acetate; hydro-cortisone 17-valerate; hydrocortisone 17-butyrate; betamethasone valerate; betamethasone dipropionate; triamcinolone acetonide; fluocinonide; clobetasol propionate; benzoyl peroxide; crotamiton; propranolol; promethazine; vitamin A palmitate; vitamin E acetate and mixtures thereof.

19. The composition according to claim 16, wherein the photoprotective agents are chosen from organic and mineral photoprotective agents.

20. A method for treating skin comprising delivering a cosmetically effective amount of at least one delta opioid receptor agonist to an area in need of such delivery.
